(19) United States
(12) Patent Application Publication
(10) Pub. No.: US 2022/0296917 A1
(43) Pub. Date: Sep. 22, 2022

(54) LIGHT THERAPY DEVICE FOR NON-INVASIVE STIMULATION OF THE HUMAN BRAIN

(71) Applicant: REBALANCE TECH CORP., Wilmington, DE (US)

(72) Inventors: Philippe Avice, Benahavis (ES); Corinne Klucik, Benahavis (ES)

(21) Appl. No.: 17/206,346

(22) Filed: Mar. 19, 2021

(51) Int. Cl. A61N 5/06

(52) U.S. Cl. CPC A61N 5/0613; A61N 2005/0652; A61N 2005/0663; A61N 2005/0664

(57) ABSTRACT

A light therapy device for stimulating the human brain includes a seat for supporting the body of a user. The device further includes a light array having individual light emitting devices positioned above the seat. The device further includes a controller for controlling the light array and generating instructions for the individual light emitting devices to produce light. The seat maintains the body of the user in a zero gravity position. The light array is located at a position above the seat to allow the light array to illuminate the face of the user.

### FIELD OF THE INVENTION

This disclosure relates to a light therapy device for non-invasive stimulation of the brain of a mammal, in particular of a human being.

### BACKGROUND OF THE INVENTION

The present disclosure relates to a light therapy device, which is adapted to stimulate the human brain by combining different light therapy protocols. The objectives of these light therapy protocols are to fight stress and to prevent health problems for human beings. The device according to the disclosure is specifically adapted to provide brain stimulation by combining chromotherapy protocols, pulsed luminotherapy protocols and dynamic light Mandalas. The combination of the three therapeutic treatment can be referred to as Synchromotherapy™.

Chromotherapy consists in exposing a human being to the different colors of the color spectrum for therapeutic purposes. The basic principles of chromotherapy are based on the knowledge that every color has therapeutic and psychological properties, as the human body depends biochemically on light. For instance, the color red is the color of force, health and vitality. The color red stimulates and activates the blood circulation and has a stimulating effect on the liver. By exposing the human body to colors, psychological and therapeutic benefits can be obtained, these benefits playing an important role in the well-being, and the quality of life of a human being.

Luminotherapy, or light therapy, is known as a treatment for seasonal affective disorder. To compensate for the lack of daylight, a human being is exposed to daylight or an equivalent form of light. Luminotherapy can be used as a treatment for wintertime related disorders, such as delayed sleep phase disorders.

The term ‘Mandala’ refers to a circular art made up of various patterns and colors. Mandalas are used in meditation and are part of the tradition in Buddhism. The Mandala will have an effect on the human brain through stimulation of the human brain by the combination of the pattern and colors used in the Mandala. The effect of a Mandala on the human brain can be measured by means of an EEG. There are different ranges of brain waves that an EEG device can detect. The main brain waves are known as Theta (4-7 Hz), Alpha (7-14 Hz) and Beta (14-30 Hz) brainwaves. Theta brainwaves represent a calm and peaceful state of mind. Alpha brainwaves represent a relaxed and learning state of mind. Beta brainwaves represent an agitated state of mind. The EEG can therefore provide an indication of the effect of a Mandala on the state of mind of a human being.

The device according to the disclosure is specifically adapted to use the combination of the above-mentioned chromotherapy, luminotherapy and Mandalas to obtain beneficial therapeutical effects on a human being.

It is further noted that in the human body, the Autonomous Nervous System (ANS) regulates various automatic processes in the human body like, for instance, the heart rate, the digestion and thermoregulation. The term ‘automatic processes’ is used to refer to the fact that those processes take place, without the human being providing distinct instructions to the different parts of the body, to activate those processes.

The ANS is composed of two subsystems with opposing actions: firstly, the sympathetic nervous system's main function is to activate the physiological changes that occur during the fight-or-flight response. The fight-or-flight response, or hyperarousal and also called the ‘acute stress response’, is a physiological reaction that occurs in response to a perceived harmful event, such as an attack or threat to survival.

Secondly, the parasympathetic system has as main function to activate the rest and digests response and triggers internal maintenance functions of the body, fostering regeneration and recovery.

One could describe the sympathetic nervous system as having a function of ‘accelerator’ of processes in the human body and the parasympathetic nervous system as having the function of a ‘brake’ for those processes.

Modern life puts human beings under increased pressure. The high level of stress on human beings can affect the parasympathetic nervous system. In this context, a balanced ANS is a direct indicator of good health and low stress levels. Therefore, it is important to strengthen the parasympathetic nervous system to achieve an optimum state of balance in the ANS.

The aim of the device according to the disclosure is to provide brain stimulation, which can actively help a human being to improve the balance in the ANS.

### SUMMARY OF THE INVENTION

Exemplary embodiments are defined by the claims.

According to a first aspect, the invention relates to a light therapy device for stimulating the human brain, the device comprising:
a seat for supporting the body of a user,
a light array, comprising a plurality of individual light emitting devices, positioned above the seat, and
a controller for controlling the light array, configured to generate instructions for the individual light emitting devices to thereby emit light by means of said plurality of light sources,
   wherein the seat is adapted to maintain the body of a user in a zero gravity position and wherein the light array is fixed at a predetermined position above the seat to allow the light array to illuminate the face of a user positioned on the support.

According to an exemplary embodiment the light emitting devices are configured to emit multicolor light.

According to an exemplary embodiment the light array comprises a plurality of LED devices.

According to an exemplary embodiment the light array has a circular shape.

According to an exemplary embodiment the controller is configured to control the color of the light produced by plurality of light emitting devices.

According to an exemplary embodiment the controller is configured to control the switch-on frequency of the light emitting devices in the light array.

According to an exemplary embodiment the controller is configured to control the intensity of the light emitting devices in the light array.

According to an exemplary embodiment the light array is positioned at a distance of 80-120 cm with respect to the seat, preferably between 90 and 110 cm, with more preference at a distance of 102 cm.

According to an exemplary embodiment the seat is configured to support the body of a user in a near horizontal position, wherein the shoulders the heart, the knees and the feet of the body are essentially aligned.

According to a further aspect, the invention relates to a light therapy device for stimulating the human brain, the device comprising:
a seat for supporting the body of a user in a zero-gravity position,
a light array, comprising a plurality of light emitting devices, fixed at a predetermined position above the seat, to allow the light array to illuminate the face of a user positioned on the support, and
a controller for controlling the light array, configured to generate instructions for the individual light emitting devices to thereby produce light by means of said plurality of light emitting devices,
   wherein the controller is configured to control the color, the intensity and the switch-on frequency of the light produced by means of the individual light sources in the plurality of light emitting devices.

According to a yet a further aspect, the invention relates to a light therapy device for stimulating the human brain, the device comprising:
a seat for supporting the body of a user in a zero-gravity position,
a light array, comprising a plurality of light emitting devices, fixed at a predetermined position above the seat, to allow the light array to illuminate the face of a user positioned on the support, and
a controller for controlling the light array, configured to generate instructions for the individual light emitting devices to thereby produce light by means of said plurality of light emitting devices, the controller being configured to control the color, the intensity and the switch-on frequency of the light produced by means of the individual light sources in the plurality of light emitting devices,
wherein the light therapy device is configured to create alternating light effects, to thereby stimulate the human brain.

### BRIEF DESCRIPTION OF THE DRAWINGS

FIG. 1 shows an exemplary embodiment of the light therapy device according to the disclosure,

FIG. 2 shows the light therapy device according to FIG. 1 with a user positioned on the seat of the light therapy device,

FIG. 3 shows the lower face of the upper part of the light therapy device according to FIGS. 1 and 2, comprising a light array, and

FIG. 4 shows the light array of FIG. 3 in more detail.

### DESCRIPTION OF PREFERRED EMBODIMENTS

The subject matter of select exemplary embodiments is described herein to meet statutory requirements. The description itself is not intended to necessarily limit the scope of the claims. Rather, the claimed subject matter might be embodied in other ways to include different components similar to the ones described in this document, in conjunction with other present or future technologies. Terms should not be interpreted as implying any particular order among or between steps herein disclosed.

In the present disclosure, the term ‘zero-gravity position’ is used to indicate the position of the human body positioned on the support of the light therapy device according to the disclosure.

The zero-gravity (or Zero-G) position is a neutral body posture initially developed by NASA. It is defined as being in a state or condition which reduces the strain put on a human body and which facilitates blood circulation in the body and reduces stress on the joints. In the context of this disclosure the, the zero-gravity position is a near horizontal position wherein the shoulders, the heart, the knees and the feet of a human being are essentially aligned. The back and the upper legs of a human being and respectively the upper legs and the lower legs form obtuse angles to relieve stress and optimize blood circulation.

In the present disclosure, reference is made to a light array comprising a plurality of light sources, for instance LED devices. The light array according to the disclosure has the form of an iris. This means that the plurality of light sources together forms light element, wherein the individual light sources are arranged in longitudinal light elements, which are positioned as spokes of a wheel to form an essentially circular light array.

In the present disclosure the term dynamic light Mandala refers to a circular light pattern obtained by means of the light array, comprising sequences of defined colors associated with defined frequencies.

With reference to FIGS. 1-4, a light therapy device 1 is described in accordance with an exemplary embodiment. The light therapy device 1 comprises a lower part 2, which is adapted to be positioned on a support 70. The top side of the lower part 2 is shaped to form a seat 20.

In the example of FIG. 1, the seat 20 is covered with a mattress 40 comprising a material adapted to support the human body. The mattress 40 can comprise a quilted or similarly fastened case, of a heavy cloth, leather of a synthetic material. The case comprises material such as a foam, foam rubber, cotton, or any other adapted material or combinations thereof to provide a comfortable support for the human body.

Preferably, the seat material used for covering the mattress has specific characteristics to allow the seat material to be easily cleaned, disinfected and sterilized.

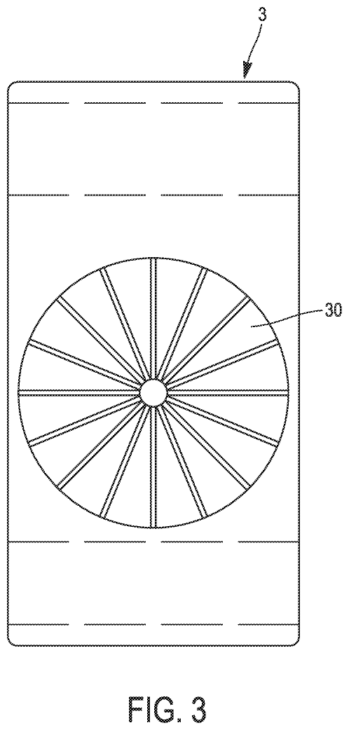

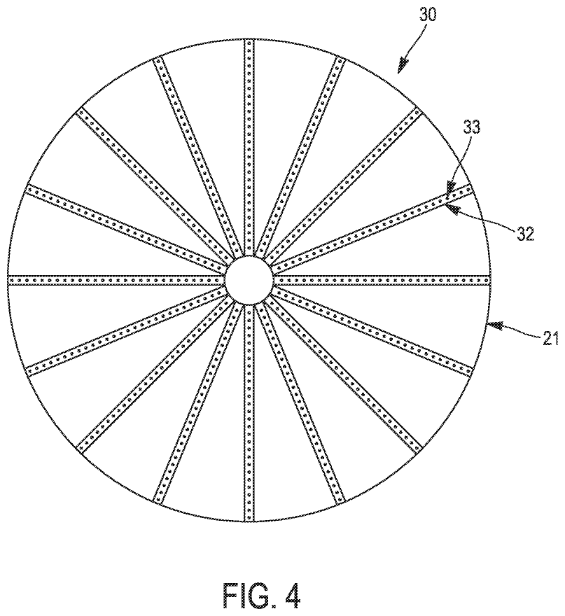

As explained in more detail with reference to FIG. 3, the seat is shaped to allow a user to be seated in a zero-gravity position.

The light therapy device 1 further comprises an upper part 3, which extends above the seat 20. The upper part 3 comprises a light array 30. The shape of the light array 30 is shown in detail in FIGS. 3 and 4.

The upper part 3 is connected to the lower part 2 by means of a connection 4.

The upper part 3 is positioned in relation to the lower part 2 to ensure that the light array 30 is located at a predetermined position in relation to the seat 20 and, more particularly, to ensure that the light array is located at a predetermined position in relation to a user's face. In FIG. 1 the face of a user is schematically represented by means of the dashed line 65.

The specific distance between the seat 20 and the light array 30 is determined such that the face 65 of a user can be properly illuminated using the light array 30. In FIG. 1, the reference number 6 indicates this specific distance between the light array 30 and the face of the user.

The size and shape of the seat 20 and the light array 30 and their angular position are adapted to allow a user to concentrate on the light array 30 and in particular the center of the light array 30 with the least effort possible.

The dotted line 6 represents the neutral position of the user's eyes, which is directed to the center of the light array. The aim of the mutual position between the seat 20 and the light array 30 is to occupy the visual perception space of the user.

The light therapy device 1 further comprises a controller 5. In the example of FIG. 1 the controller is positioned in the lower part 2 of the light therapy device 1. It should be understood that alternatively the controller 5 could be positioned in the upper part 3 of the light therapy device 1 or could be positioned remotely from the light therapy device 1.

The controller 5 is connected to the light array 30 and is adapted to provide instructions to the light array 30. The connection between the controller 5 and the light array 30 is schematically indicated with connection 51. This connection 51 can be obtained by means of a wire or can be wireless. For a wireless connection an adapted protocol, can be selected, such as Bluetooth.

As an example, the light therapy device 1 could use a RJ45 Wired connection, using a standard Serial protocol.

The light array 30 comprises a plurality of light sources, typically a plurality of Light Emitting Diodes (LEDs). The controller 5 is adapted to provide instructions to the light array 30 thereby controlling the color, the intensity and the switch-on frequency of the individual light sources in the light array 30. The combination of these instructions provides a specific light effect, which is used for therapeutic objectives.

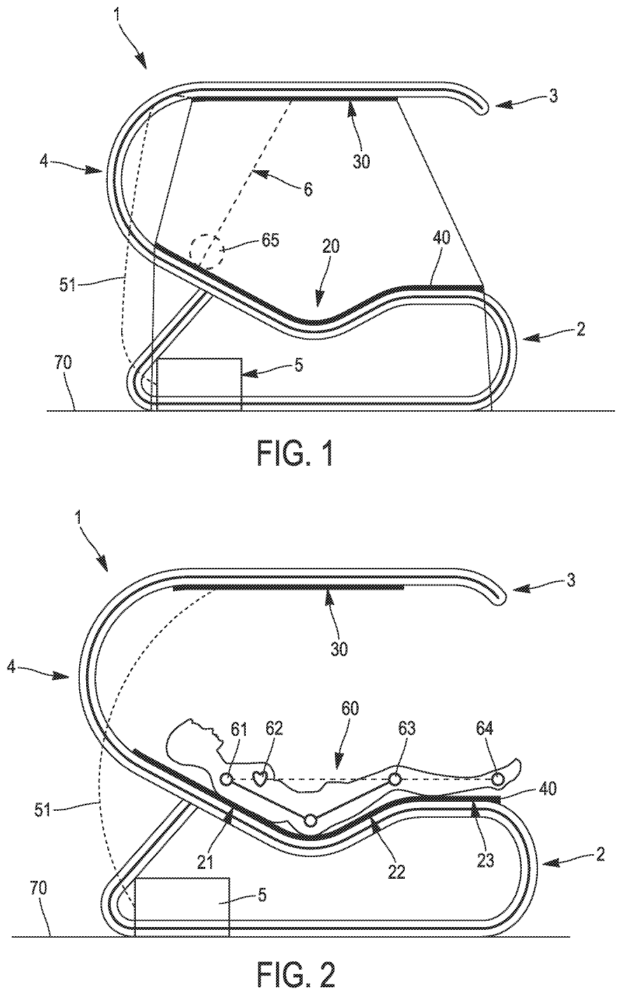

FIG. 2 shows the light therapy device 1, with a user 60 positioned on the seat 20. The seat 20 has a size and shape to allow the user 60 to be in a zero-gravity position. The main purpose of this zero-gravity position is to limit strain on the different body parts of the user 60 and to facilitate blood circulation in the body of the user 60. According to the disclosure, the zero-gravity position imposed by the seat will allow the user 60 to be in a near horizontal position, wherein the shoulders 61, the heart 62, the knees 63 and the ankles 64 of the user 60 are essentially aligned. As shown in FIG. 2 the back and the upper legs of the user 60 and, respectively, the upper legs and the lower legs of the user 60 form obtuse angles.

The seat 20 comprises three main elements, which are angled with respect to each other. The backrest 21 of the seat 20 is adapted to support the back of the user 60. The upper leg rest 22 of the seat 20 is adapted to support the upper legs of the user 60. The lower legs support 23 is adapted to support the lower legs of the user 60.

To allow the user 60 to maintain the zero-gravity position, the backrest 21 is inclined with respect to the support 70 under an angle between 140 and 160 degrees, preferably between 145 and 155 degrees, with more preference of 152 degrees.

To allow the user 60 to maintain the zero-gravity position, the upper leg rest 22 is inclined with respect to the support 70 under an angle between 140 and 160 degrees, preferably between 145 and 155 degrees, with more preference of 150 degrees. The backrest 21 and the upper leg rest 22 define an obtuse angle between 110 and 130 degrees, preferably between 115 and 125 degrees, with more preference of 122 degrees.

To allow the user 60 to maintain the zero-gravity position, the lower leg rest 23 is inclined with respect to the support 70 under an angle between −10 and 10 degrees, preferably between −5 and 5 degrees, with more preference of 0 degree.

The user 60 will be in a zero-gravity position and in this position will be with its face at a determined distance 6 from the light array 30. This predetermined distance 6 is between 80 and 120 cm, preferably between 90 and 110 cm, with more preference is 102 cm.

FIG. 3 shows the bottom face of the upper part 3 of the light therapy device 1. The upper part 3 comprises a light array 30, which has the form of an iris or disk. The independent light sources of the light array 30 from linear strips that are positioned as the spokes of a wheel to form the disk shaped light array 30.

The light array 30 is shown in more detail in FIG. 4. The light array 30 is formed by a total of 16 light strips 32. Each of the strips 32 comprises a plurality of individual light sources 33, in particular LEDs. According to the example of FIG. 4 each of the strips 32 comprises in total 28 independent LEDs.

For the LEDs, typically multicolor LEDs are used. For instance, Standard RGB

LEDs can be used having the three components light components RGB controllable independently with a total of 24/32 bit, providing: “Truecolor” depth.

The light array 30 is adapted to provide a number of light effects, which are used in light therapy for a user 60. To obtain an optimum result, the diameter of the light array 30 is in the order of 90 cm-120 cm, preferably between 95 cm and 110 cm, with more preference between 105 and 108 cm.

Use of the Light Therapy Device

The light therapy device 1 according to the disclosure is used to expose a user to the specific light produced by the light array 30.

With the light therapy device 1, it is a first option to use the controller 5 to provide instructions for the LEDs 33 in the light strips 32 of the light array 30 to produce a specific color. Because of the predetermined distance between the light array 30 and the face 65 and the body of a user 60 positioned on the seat 20, the user 60 will be exposed to this specific color.

In this manner the light therapy device 1 according to the disclosure can be used for chromotherapy purposes.

As an example, during a Synchromotherapy session, a light mandala formed by means of the light array 30 will be synchronized with audio guidance to perform breathing and meditation exercises. The visual perception space of the user will be occupied by the light mandala, the synchronism will help the user to follow the exercises. The rhythm is based on a respiratory rate known as cardiac coherence.

The controller 5 can further be used to provide instructions to the LEDs 33 in the light strips 32 of the light array 30 to create light beams with a certain color and a certain intensity. Moreover, the frequency wherein the light beams can be produced can be controlled with the controller. This allows exposing a user 60 to light beam with a variety of intensities and frequencies.

The colors follow the Synchromotherapy session, and they are defined by the theme of the session and the actual exercise.

In this manner, the light therapy device can be used for luminotherapy protocols, in particular pulsed luminotherapy protocols.

Additionally, the controller can be used to provide instructions to the LEDs 33 in the light strips 32 of the light array 30 to create colorful, circular images. The presence of the plurality of light sources 33, of each of which the color can be adjusted independently, allows for a large variety of option to create the colorful, circular images.

In order words, in this manner the light therapy device 1 can be used to expose a user to dynamic light Mandalas.

The mandala is composed of predefined colors and pattern chosen in adequation with the theme of the exercise.

It will be understood that the presence of the light array 30 and the instructions that can be generated by the controller 5 to control the individual light sources 33, allow a combination of the three above-mentioned light therapies: chromotherapy protocols, pulsed luminotherapy protocols and dynamic light Mandalas. The combination of the three light therapies can be referred to as Synchromotherapy™.

It has been established, that the result of the combination of the three mentioned therapies is greater than the sum of the partial results produced by each of the three therapies individually.

The beneficial effects that can be obtained by means of the light therapy device 1 according to the disclosure are further enhanced because of the zero-gravity position that is imposed on the user 60 during the operation of the device 1. The relieve of stress on the body of the user and the optimized blood circulation obtained by the zero-gravity position is a further accelerator for the positive effects of the different light therapies on the user 60.

The above described Synchromotherapy™ can be used with the aim of obtaining the following effects on a human being:
Chronic stress reduction
Decreased blood pressure of hypertensive people
Better-quality sleep
Stronger immune system
A restored, enhanced level of vitality
Increased recovery speed
Increased ability to concentrate and memorize
Increased brain agility
Telomere protection and lengthening (anti-ageing effect)

The light therapy device 1 can be used for Synchromotherapy™ as described above. During the operation of the device 1, the human brain can further be stimulated, thereby further enhancing the beneficial influence of the device 1, by asking a user to concentrate on a number of exercises and/or techniques.

These exercises and techniques include:
Breathing exercises and techniques chromorythm-based
Guided mental imagery
Cardiac coherence
Mindfulness training
Full body chromotherapy protocol
Sound therapy protocol
Binaural synchrotherapy protocol
Synchromotherapy protocol
Advanced meditation exercises

### EXAMPLES

### Example 1: EEG Study

A EEG study was conducted under conditions that could be described as “stressful” or at least as “not very relaxing”. The test subjects were fitted with a large number of sensors to monitor the reaction of the subjects body to the stimulation generated by the light therapy device 1. In total fifteen subjects were tested, including ten top-level athletes. The tests were conducted in a space where the researchers were physically present, along with the requisite computer and electronic equipment for taking measurements.

In total a test with a length of 38-minutes was completed. The test session comprising six specific programs/sequences was deliberately interrupted six times to allow calibration of the measurements.

The hypothesis was that the panel of high-level athletes could overcome this “not very relaxing” environment more easily as they are accustomed to putting their body in circumstances of stressful nature. The use of rigorous protocols is similar in every respect to that applied during studies with NASA astronauts on the International Space Station. The various stimulations and exercises conducted during the test session yielded and confirmed the following results:

1—The subject showed a strong increase in alpha wave amplitude and duration, particularly significant at 11 Hz. This amplitude was maintained even after the test sessions ended.
2—A sharp increase in theta wave rhythm, associated with robust stability throughout the sessions.
3—These two major characteristics were observed whether participants' eyes were open or closed This three-dimensional mental pattern observed from the first test session is reminiscent of characteristics more commonly observed in studies carried out on expert meditators.

### Example 2: Effect of the Light Therapy Device on Two Populations with Different Stress Levels: Impact on Physiological Variables and Sleep Quality

According to the prior art comparative performances of different therapies and meditative practices are correlated to the reduction in blood pressure of hypertensive people (HBP—High Blood Pressure). See for instance, Dickinson (Dickinson et al. Lifestyle interventions to reduce raised blood pressure: a systematic review of randomized controlled trials. 2006 February; 24(2):215-33.)

The average observed in these studies corresponds to a decrease of −5.0 mmHg in systolic blood pressure and −2.8 mmHg in diastolic blood pressure.

When observing the greater performance (Patel 1988: Patel C, Marmot M. Can general practitioners use training in relaxation and management of stress to reduce mild hypertension? BMJ 1988; 296: 21-24.), the average value recorded is a diminution of −14.5 mmHg in systolic blood pressure and −6.9 mmHg in diastolic blood pressure.

Concerning the use of the light therapy device according to the disclosure, the average performance is −19.4 mmHg in systolic blood pressure and −8.5 mmHg in diastolic blood pressure for hypertensive people (page 20—HBP—High Blood Pressure). In summary, the light therapy device 1 significantly outperforms the average observed in previous studies and we largely exceed the greatest performance of the literature review.

It may be noted that this greatest performance (Patel 1988: Patel C, Marmot M. Can general practitioners use training in relaxation and management of stress to reduce mild hypertension? BMJ 1988; 296: 21-24.) obtained these results thanks to «active drugs», unlike the results obtained using of the light therapy device according to the disclosure which results have been achieved without drugs.

The various automatic processes of our body (heart rate, digestion, thermoregulation, . . . ) are regulated by the Autonomous Nervous System (ANS).

The ANS is composed of two subsystems with opposing actions: the sympathetic system (accelerator) is activated during a fight or as a response to a threat, but is too prevalent in a state of chronic stress. The second one, the parasympathetic system (brake), on the other hand triggers the internal maintenance functions, fostering regeneration and recovery.

One of the main objectives of the light therapy device 1 according to the disclosure is to strengthen the parasympathetic branch (the brake) often affected by modern life to achieve an optimum state of balance in the autonomic nervous system. In this context, a balanced ANS is a direct indicator of a good health and low stress.

In this study, the sessions were performed over a period of one month and enabled the hypertensive population (HBP) to reduce their resting heart rate by an average of almost 10 beats per minute. A study on the effect on sleep was performed. In this sleep study, through all the analysis and control methods, witnesses not only showed a strong improvement of the quality of sleep but also promptness in obtaining regenerating sleep.

According to the studies and clinical tests brought to our attention by the panel of experts, it appeared highly unlikely to induce telomere lengthening over a period of less than three months. In the tests with the light therapy device according to the invention, covering 10 sessions over a period of one month, revealed telomere lengthening, which seems to be a first in such a short time. In addition to the notable and demonstrated effectiveness of the light therapy device 1 in terms of health prevention and overall health improvement, it is the rapidity in obtaining these results that distinguishes the performance of the light therapy device according to the disclosure.

1. A light therapy device for stimulating the human brain, the device comprising:
a seat for supporting the body of a user,
a light array, comprising a plurality of individual light emitting devices, positioned above the seat, wherein the individual light emitting devices are arranged in linear strips that are positioned as spokes of a wheel, and
a controller for controlling the light array, configured to generate instructions for the individual light emitting devices to thereby emit light by means of said plurality of light sources,
wherein the seat is adapted to maintain the body of a user in a zero gravity position and wherein the light array is fixed at a predetermined position above the seat to allow the light array to illuminate the face of a user positioned on the support.

2. Light therapy device according to claim 1, wherein the light emitting devices are configured to emit multicolor light.

3. Light therapy device according to claim 1, wherein the light array comprises a plurality of LED devices.

4. Light therapy device according to claim 1, wherein the light array has a circular shape.

5. Light therapy device according to claim 1, wherein the controller is configured to control the color of the light produced by plurality of light emitting devices.

6. Light therapy device according to claim 1, wherein the controller is configured to control the switch-on frequency of the light emitting devices in the light array.

7. Light therapy device according to claim 1, wherein the controller is configured to control the intensity of the light emitting devices in the light array.

8. Light therapy device according to claim 1, wherein the light array is positioned at a distance of 80-120 cm with respect to the seat.

9. Light therapy device according to claim 1, wherein the seat is configured to support the body of a user in a near horizontal position, wherein the shoulders the heart, the knees and the feet of the body are essentially aligned.

10. Light therapy device for stimulating the human brain, the device comprising:
a seat for supporting the body of a user in a zero-gravity position,
a light array, comprising a plurality of light emitting devices, fixed at a predetermined position above the seat, to allow the light array to illuminate the face of a user positioned on the support, seat, wherein the light emitting devices are arranged in linear strips that are positioned as spokes of a wheel, and
a controller for controlling the light array, configured to generate instructions for the individual light emitting devices to thereby produce light by means of said plurality of light emitting devices, wherein the controller is configured to control the color, the intensity and the switch-on frequency of the light produced by means of the individual light sources in the plurality of light emitting devices.

11. Light therapy device for stimulating the human brain, the device comprising:
a seat for supporting the body of a user in a zero-gravity position,
a light array, comprising a plurality of light emitting devices, fixed at a predetermined position above the seat, to allow the light array to illuminate the face of a user positioned on the support seat, wherein the light emitting devices are arranged in linear strips that are positioned as spokes of a wheel, and
a controller for controlling the light array, configured to generate instructions for the individual light emitting devices to thereby produce light by means of said plurality of light emitting devices, the controller being configured to control the color, the intensity and the switch-on frequency of the light produced by means of the individual light sources in the plurality of light emitting devices, wherein the light therapy device is configured to create alternating light effects, to thereby stimulate the human brain.

12. Light therapy device according to claim 8, wherein the light array is positioned at a distance of 90-110 cm with respect to the seat.

13. Light therapy device according to claim 12, wherein the light array is positioned at a distance of 102 cm with respect to the seat.

14. Light therapy device according to claim 1 wherein the controller is configured to control the light array to generate a dynamic light mandala.

15. Light therapy device according to claim 10 wherein the controller is configured to control the light array to generate a dynamic light mandala.

16. Light therapy device according to claim 11 wherein the controller is configured to control the light array to generate a dynamic light mandala.
